(19) United States
(12) Patent Application Publication
(10) Pub. No.: US 2009/0270310 A1
(43) Pub. Date: Oct. 29, 2009

(54) Process for the preparation of a nutrient formulation

(75) Inventor: Susan Kay Whyte, Dalkeith (AU)

Correspondence Address: BROWDY AND NEIMARK, P.L.L.C.;624 NINTH STREET, NW, SUITE 300, WASHINGTON, DC, 20001-5303, US

(73) Assignee: Chemstop Pty Ltd, Dalkeith (AU)

(21) Appl. No.: 12/457,086

(22) Filed: Jun. 1, 2009

Related U.S. Application Data
(62) Division of application No. 10/503,066, filed on Mar. 18, 2005, now abandoned.

(30) Foreign Application Priority Data
Jan. 31, 2002 (AU) AU PSO198

(51) Int. Cl. A61K 38/16; A61K 31/7088; A61K 38/00; A61K 31/56; A61K 31/20; A61P 11/08

(52) U.S. Cl. 514/8; 514/44 R; 514/12; 514/169; 514/558

(57) ABSTRACT

The present invention relates to a process for increasing the efficacy and/or bioavailability of a nutrient formulation or composition for the treatment and/or prevention of inflammatory processes associated with airway diseases such as asthma. In particular, the invention relates to a method of treating an airway disease in a subject in need of such treatment, comprising the step of administering a nutrient formulation or composition which comprises one or more components which have been agitated such that a harmonic of between 20 to 50 Hz has been produced, in an amount effective to treat said disease.

### FIELD OF THE INVENTION

The present invention relates to a process for increasing the efficacy and/or bioavailability of a nutrient formulation or composition for the treatment and/or prevention of inflammatory processes associated with airway diseases such as asthma. In particular, the invention relates to a process for increasing the efficacy and/or bioavailability of a nutrient formulation or homeopathic composition comprising the step of agitating one or more components of said formulation or composition so that a specific harmonic is obtained.

### BACKGROUND OF THE INVENTION

It is well appreciated by those skilled in the art that many of the modern therapeutics used to treat diseases as diverse as cancer, inflammation and cardiac conditions have limited efficacy and/or bioavailability in vivo. This is despite these therapeutics having demonstrated exceedingly good efficacy in in vitro bioassays and the like. To date, most, if not all research to increase the efficacy and/or bioavailability of therapeutics has been directed towards enhancing the cellular uptake and/or increasing residency time of the therapeutic. However, while this research has produced some improvement in the efficacy and/or bioavailability of therapeutics in vivo, the levels experienced are insufficient. Accordingly, there is still a need to increase the efficacy and/or bioavailability of therapeutics in vivo.

Airway diseases including cystic fibrosis, asthma, chronic obstructive pulmonary disease, bronchitis, and other airway diseases characterised by an inflammatory response are particular diseases where the efficacy and/or bioavailability of therapeutics has been poor. Asthma in particular is one of the most common diseases in industrialised countries, and in the United States and accounts for about 1% of all health care costs (K. Weiss et al., New Eng. J. Med. 326, 862-6 (1992)). An alarming increase in both the prevalence and mortality of asthma over the past decade has been reported (Asthma-United States, 1980-1990, MMWR 41:733-735 (1992); Wilson J W and Robertson C F (2002), Med. J. Austral. 177 (6):288-289), and occupational asthma is predicted to be the pre-eminent occupational lung disease in the near future (M. Chan-Yeung and J. Malo, European Resp. J. 7:346-71 (1994)).

It has been shown that asthma is triggered by chemicals which can cause inflammatory responses in the airways. Particulate air pollutants may also cause the anti-oxidant defence system to be activated (Blomberg, 2000, Clin Exp Allergy. 30:310-7). It has also been shown that serum and red blood cell anti-oxidant states are lower in patients with bronchial=asthma (Vural & Uzun, 2000, Can Respir J. 7:476-80). It has also been shown that in asthmatic patients there is a reduction of platelet GSH activity. This suggests that these patients have a diminished capacity to restore part of the anti-oxidant defences and that anti-oxidants from diet alone are not adequate to restore normal anti-oxidant levels (Picado et al., 2001, Allergy 56:43-9)

The applicant has now surprisingly found that compositions for the treatment of airway disease and in particular asthma, may be enhanced with respect to efficacy and/or bioavailability by using specific agitation methods which produce particular harmonics such that anti-oxidant levels are restored.

### SUMMARY OF THE INVENTION

A first aspect of the invention provides a method of treating an airway disease in a subject in need of such treatment, comprising the step of administering a nutrient formulation or composition which comprises one or more components which have been agitated such that a harmonic of between 20 to 50 Hz has been produced, in an amount effective to treat said disease.

A second aspect of the present invention provides a nutrient formulation or composition useful for treating an airway disease in a subject in need of such treatment, comprising ascorbic acid, magnesium and selenomethionine and a pharmaceutically acceptable carrier, wherein one or more components have been agitated such that a harmonic of between 20 to 50 Hz has been produced, together in an amount effective to treat said disease.

A method of producing a nutrient formulation or composition useful for treating an airway disease in a subject in need of such treatment, said formulation or composition comprising vitamins, trace elements and probiotic bacteria said method comprising the step of agitating at least one component of said formulation or composition such that a harmonic of between 20 to 50 Hz is produced.

Accordingly, the present invention provides a novel process and nutrient formulation or composition for treating an airway disease. This and other aspects are achieved in whole or in part by the present invention.

The foregoing and other aspects of the present invention are explained in greater detail in the specification below.

### BRIEF DESCRIPTION OF THE FIGURES

FIG. 1 is a diagram of an experimental apparatus used in Example 1.

### DETAILED DESCRIPTION OF THE INVENTION

The practice of the present invention employs, unless otherwise indicated, conventional chemistry and pharmacology within the skill of the art. Such techniques are well known to the skilled worker, and are explained fully in the literature. See, e.g., Coligan et al., “Current Protocols in Protein Science” (1999) Volume I and II (John Wiley & Sons Inc.); and Bailey, J E and Ollis, D F, Biochemical Engineering Fundamentals, McGraw-Hill Book Company, New York, 1986.

Before the present methods are described, it is understood that this invention is not limited to the particular materials and methods described, as these may vary. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “an,” and “the” include plural reference unless the context clearly dictates otherwise. Thus, for example, a reference to “a compound” includes a plurality of such compounds, and a reference to “an harmonic” is a reference to one or more harmonics, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

All publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

The present invention relates to methods of treating “airway diseases” and in particular methods of increasing the efficacy and/or bioavailability of a “nutrient formulation” or “homeopathic composition” and a method of producing such nutrient formulations or composition. The terms “formulation” and “composition” as used herein are interchangeable and includes any substance, or agent that can be used to treat airway diseases as defined herein.

Examples of airway diseases that can be treated by the method of the present invention include cystic fibrosis, asthma, chronic obstructive pulmonary disease, bronchitis, and other airway diseases characterised by an inflammatory response. Treatment of airway inflammation is also provided in accordance with the present invention, including inflammation with or without (i.e., free of) asthma.

As used herein, the term “treat” or “treating” an airway disease refers to a treatment which decreases the likelihood that the subject administered such treatment will manifest symptoms of the airway disease.

The term “subject” as used herein refers to any vertebrate species which suffers from airway disease. The methods of the present invention are particularly useful in the treatment of warm-blooded vertebrates. Thus, in a preferred embodiment, the invention concerns mammals and birds.

In one preferred embodiment the present invention is concerned primarily with the treatment of human subjects, but can also be employed for the treatment of other mammalian subjects, such as dogs, cat, livestock, primates and horses, for veterinary purposes.

Thus, provided is the treatment of mammals such as humans, as well as those mammals of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economical importance to humans. Thus, provided is the treatment of livestock, including, but not limited to, domesticated swine (pigs and hogs), ruminants, horses, poultry, and the like.

The formulation or composition preferably includes an active agent. As used herein, the term “active agent” refers to an agent which possesses therapeutic or prophylactic properties in vivo, for example when administered to a subject. The term “active agent” also includes other (non-active) substances, which may, for example, be administered together with or combined with the active agent to aid administration. Examples of suitable therapeutic and/or prophylactic active agents include proteins, such as hormones, antigens, and growth factors; vitamins and minerals; probiotic bacteria; nucleic acids; and smaller molecules, such as antibiotics, steroids, and decongestants.

The active agent can include organic molecules such as a drug, peptide, protein, carbohydrate (including monosaccharides, oligosaccharides, and polysaccharides), nucleoprotein, mucoprotein, lipoprotein, synthetic polypeptide or protein, or a small molecule linked to a protein, glycoprotein, steroid, nucleic acid (any form of DNA, including cDNA, or RNA, or a fragment thereof), nucleotide, nucleoside, oligonucleotides (including antisense oligonucleotides), gene, lipid, hormone, vitamin, including vitamin C and vitamin E, minerals and elements such as magnesium, selenium or combinations thereof.

Representative therapeutic active agents include antioxidants, chemotherapeutic agents, steroids (including retinoids), hormones, antibiotics, antivirals, antifungals, antiproliferatives, antihistamines, anticoagulants, antiphotoaging agents, melanotropic peptides, nonsteroidal and steroidal anti-inflammatory compounds. Other non-limiting examples of active agents include anti-infectives such as nitrofurazone, sodium propionate, antibiotics, including penicillin, tetracycline, oxytetracycline, chlorotetracycline, bacitracin, nystatin, streptomycin, neomycin, polymyxin, gramicidin, chloramphenicol, erythromycin, and azithromycin; sulfonamides, including sulfacetamide, sulfamethizole, sulfamethazine, sulfadiazine, sulfamerazine, and sulfisoxazole, and anti-virals including idoxuridine; antiallergenics such as antazoline, methapyritene, chlorpheniramine, pyrilamine prophenpyridamine, hydrocortisone, cortisone, hydrocortisone acetate, dexamethasone, dexamethasone 21-phosphate, fluocinolone, triamcinolone, medrysone, prednisolone, prednisolone 21-sodium succinate, and prednisolone acetate; desensitizing agents such as ragweed pollen antigens, hay fever pollen antigens, dust antigen and milk antigen; decongestants such as phenylephrine, naphazoline, and tetrahydrazoline; miotics and anticholinesterases such as pilocarpine, esperine salicylate, carbachol, diisopropyl fluorophosphate, phospholine iodide, and demecarium bromide; parasympatholytics such as atropine sulfate, cyclopentolate, homatropine, scopolamine, tropicamide, eucatropine, and hydroxyamphetamine; sympathomimetics such as epinephrine; sedatives and hypnotics such as pentobarbital sodium, phenobarbital, secobarbital sodium, codeine, (α-bromoisovaleryl) urea, carbromal; psychic energizers such as 3-(2-aminopropyl) indole acetate and 3-(2-aminobutyl) indole acetate; tranquilizers such as reserpine, chlorpromayline, and thiopropazate; androgenic steroids such as methyl-testosterone and fluorymesterone; estrogens such as estrone, 17-β-estradiol, ethinyl estradiol, and diethyl stilbestrol; progestational agents such as progesterone, megestrol, melengestrol, chlormadinone, ethisterone, norethynodrel, 19-norprogesterone, norethindrone, medroxyprogesterone and 17-β-hydroxy-progesterone; humoral agents such as the prostaglandins, for example PGE₁, PGE₂ and PGF₂; antipyretics such as aspirin, sodium salicylate, and salicylamide; antispasmodics such as atropine, methantheline, papaverine, and methscopolamine bromide; antimalarials such as the 4-aminoquinolines, 8-aminoquinolines, chloroquine, and pyrimethamine, antihistamines such as diphenhydramine, dimenhydrinate, tripelennamine, perphenazine, and chlorphenazine; cardioactive agents such as dibenzhydroflume thiazide, flumethiazide, chlorothiazide, and aminotrate; nutritional agents such as vitamins, natural and synthetic bioactive peptides and proteins, including growth factors, cell adhesion factors, cytokines, and biological response modifiers.

The amount of active agent that may be combined with the carrier materials to produce a single dosage form will vary depending upon the host treated and the particular mode of administration. For example, a formulation intended for the oral administration of humans may vary from about 5 to about 95% of the total composition. Dosage unit forms will generally contain between from about 1 mg to about 500 mg of active agent.

It will be understood, however, that the specific dose level for any particular subject will depend upon a variety of factors including the activity of the specific compound employed, the age, body weight, general health, sex, diet time of administration, route of administration, rate of excretion, drug combination and the severity of the particular airway disease undergoing therapy.

In one embodiment, the nutrient formulation or composition comprises a liquid consisting of dry agents blended together. One particularly preferred nutrient formulation comprises ascorbic acid (about 250 to 350 mg, calcium (about 200 to 290 mg, magnesium (about 20 to 25 mg, zinc (about 12 to 25 mg, selenomethionine (about 0.02 to 0.1 mg, Na bicarbonate (about 330 to 400 mg, boron from a homeopathic source between 1× and 1, and probiotic bacteria between 1 to 10¹¹ cfu per gm blended together with between 400 ml to 1000 ml water and 2% of a suitable “non toxic surfactant”. The term “non toxic surfactant” may include lecithin or glycerol, potassium sorbate and ethanol. The method of blending of the dry agents, water and surfactant is not essential and any standard techniques used in the art may be employed.

The preferred formulation or composition may also include a nutritionally acceptable soluble magnesium salt, for example in the form of magnesium aspartate or orotate. Other additives include soluble calcium salt, ascorbic acid derivative, for example calcium citrate, orotate or carbonate, sodium, potassium, magnesium aspartate or orotate, zinc ascorbate or picolinate or aspartate or oxide; ascorbic acid, or as zinc amino acid chelate, boron, selenomethionine as well as pharmaceutically acceptable buffering salt such as, for example, sodium bicarbonate.

The active agent(s) of the formulation or composition of the invention may also be agitate with any pharmaceutically acceptable carriers or diluents. The pharmaceutically acceptable carriers or diluents used will depend upon the type of active agent, route of administration and airway disease being treated. These aspects are discussed below.

Having obtained the desired liquid nutrient formulation it is then vortexed for a period between 45 and 90 minutes as described below and then agitated for 45 and 90 minutes as described below to produce a fundamental quantum harmonic of between 20 to 50 Hz.

The vortexing and agitation may be by any means capable of forming the desired harmonic as described below. Suitable means include using static mixers (Maa, et al., J. Microencapsulation 13(4):419-33 (1996)), as well as dynamic mixing means such as agitators, homogenizers, sonication, and other process equipment known in the art.

In one embodiment, the agitation is performed by blending the dry active agents together as described above with one or more pharmaceutically acceptable carriers then vortexing and agitating the nutrient formulation through a length of pipe or tubing at conditions sufficient to create the desired harmonic, i.e., enough turbulence to induce harmonic formation.

Other static devices, such as restriction plates (flow constrictors) and filters, also can be used to create the required harmonic. In a preferred embodiment, non-static mixers are used as the agitation means. As used herein, the term “non-static mixer” refers to a device having elements that freely move within a flowing stream of the fluids to be agitated. Examples of non-static mixers include non-motorised turbines and certain flow indicators, such as a ball indicator. Another example is a flow though mixer head available on a Silverson homogeniser. Non-static mixers advantageously provide more efficient agitation than that induced by turbulent flow alone, and can be less expensive than most dynamic and static mixers. These types of static and non-static mixing means can be used to enhance or replace conventional agitation techniques, such as agitators and static mixers, which may be particularly useful when the process for making the nutrient formulation or composition of the invention is operated continuously at certain production rates. Mixing in a classic static mixer relies on a number of factors, including the rate of fluid flow. Pumps or pressure controls the fluid flow rate and can vary with pump oscillations or changing pressure. The use of a non-static mixer in a continuous process can overcome these oscillations by providing additional steady mixing, resulting in a more consistent emulsion. One of skill in the art can readily optimise these mixing means to achieve the most efficient production of the desired harmonic.

Without wishing to be bound by any theory or hypothesis the applicant believes that by vortexing and agitating the nutrient formulation or composition as described herein a vortex in the nutrient formulation or composition of the invention produces small amounts of rotons depending on speed and energy of the vortex. Rotons are second generation tachyons formed in oscillating vortex (See, for example, Shatskiy, A A, J. High Energy Phys.: 11 (2001), pp. 64; Pismen, L. Phys. Rev. 2002, pp. 8). This oscillation is fundamental in producing the harmonics which are the basis of the present invention.

In one particularly preferred embodiment the vortex is between 100 mm and 250 mm Radius and has a velocity to impart of between 50 to 100 joules per second.

Calculation of the conditions to produce the specific harmonic is as follows:

˜K^{d}+Gₜ^{np}+Σ^{g}M=0

wherein:

K^{d}=Thermal Density of Fluid

Gₜ^{np}=((T+F+R)V)̂−Pi

T=temperature

Σ^{g}=harmonic mean of fluid

F=desired harmonic fluid

M=mass of fluid

R=Energy imparted to fluid

The harmonic may be measured by a Protek multifunction counter 9100 or similar frequency meter. This is done by emersing a probe into the liquid formulation after agitation has occurred. The reading is then taken of the fundamental harmonic of the agitated liquid.

In a preferred method the liquid nutrient formulation described above is vortexed at a low velocity to form a vortex in one direction of between 30-120 rpm at which point the direction of vortex is reversed until the vortex reaches a velocity of between 30-120 rpm at which point the direction of the vortex is reversed again and so repeated until a period of 45 minutes to 90 minutes is reached.

While it is possible to use any vortex machine to produce the appropriate vortex it is preferable that the system uses the kinetic energy of isotropic fluids of a range between 40,000 and 80,000 kJ.

Once the appropriate vortex has been formed in the nutrient formulation it is then agitated at a rate of between 50,000-65,000 Kj/mole at an angle of 10-90 degrees at a frequency between 0.1-100 cycles per second. During this step the solution is energized. This stage lasts between 45 to 90 minutes.

The liquid nutrient formulation is then either containerized or potentized further as follows:

1 ml of liquid nutrient formulation is diluted with 9 ml of diluent to produce 10 ml of 1× attenuation. This is then vortexed and rotated then agitated as described below where it is succussed. A further dilution of the processed ingredient can then be made as necessary by taking 1 ml of 1× attenuation which is succussed with 9 mls of diluent to produce 10 ml of 2× attenuation and so on. This may be repeated until the desired potency is achieved.

In one embodiment rather than blending the entire dry agents then vortexing and agitating the entire liquid nutrient formulation as described above it is possible to merely vortex one or more of the agents separately then blend these agents together.

The final agitated substance can be administered in the form of a solution, as an ointment or paste, as tablets, or in the form of pellets or globules of a carrier, such as lactose. Alternatively it is possible to triturate the agent with a solid carrier. Tablets or capsules may be of suitable size which are convenient for swallowing, for example about 0.2 g to about 1 g. The final substance may also be a liquid or a powder and may be added to other substances which may not be produced by this process to make a final medicine or substance.

Once the formulation or composition of the invention has been produced and has the desired harmonic it can then be formulated for administration.

The nutrient formulation or composition of the invention may be administered orally, topically, parenterally, or by inhalation spray in dosage unit formulations containing non-toxic pharmaceutically acceptable carriers, adjuvants and vehicles. The term parenteral as used herein includes subcutaneous injections, intravenous, or intramuscular.

The formulation or composition of the invention containing the active agent may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from sweetening agents, flavoring agents, coloring agents and preserving agents in order to provide pharmaceutically elegant and palatable preparations. Tablets contain the active agent in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pats. No. 4,256,108, 4,166,452 and 4,265,874, to form osmotic therapeutic tablets for control release.

Formulations for oral use may also be presented as hard gelatin capsules where in the active agent is agitate with an inert solid diluent, for example calcium carbonate, calcium phosphate or kaolin, or as soft gelatin capsules wherein the active agent is agitate with water or an oil medium, for example peanut oil, liquid paraffin or olive oil. Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate polyvinyl-pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active agent in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active agent in admixture with a dispersing or wetting agent, suspending agent and one or more preservatives. Suitable dispersing or wetting agents and suspending agents are exemplified, for example sweetening, flavoring and coloring agents may also be present.

The formulation or composition of the invention may also be in the form of oil in-water emulsions. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose or lactose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Given the nature of airway diseases as defined herein, it will be appreciated by those of skill that one particularly preferred embodiment utilises respirable particles comprising the formulation or composition of the invention. These respirable particles can be administered as a nasal formulation. In general, respirable particles range from about 0.5 to 10 microns in diameter. For nasal administration, a particle size in the range of 10-500 □m is preferred to ensure retention in the nasal cavity.

Aerosols of liquid particles comprising the formulation or composition of the invention may be produced by any suitable means, such as with a nebuliser. See, e.g., U.S. Pat. No. 4,501,729. Nebulisers are commercially available devices which transform solutions or suspensions of the active agent into a therapeutic aerosol mist either by means of acceleration of a compressed gas, typically air or oxygen, through a narrow venturi orifice or by means of ultrasonic agitation. Suitable formulations for use in nebulisers consist of the active agent in a liquid carrier, the active agent comprising up to 40% w/w, but preferably less than 20% w/w, of the formulation. The carrier is typically water or a dilute aqueous alcoholic solution, preferably made isotonic with body fluids by the addition of, for example, sodium chloride. Optional additives include preservatives if the formulation is not prepared sterile, for example, methyl hydroxybenzoate, antioxidants, flavoring agents, volatile oils, buffering agents and surfactants.

The aerosols of solid particles comprising the active agent may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder, e.g., a metered dose thereof effective to carry out the treatments described herein, is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active agent or of a powder blend comprising the active agent, a suitable powder diluent, such as lactose, and an optional surfactant. The active agent typically comprises from 0.1 to 100 w/w of the formulation.

A second type of illustrative aerosol generator comprises a metered dose inhaler. Metered dose inhalers are pressurised aerosol dispensers, typically containing a suspension or solution formulation of the active agent in a liquefied propellant. During use these devices discharge the formulation through a valve adapted to deliver a metered volume, typically from 10 to 150 □l, to produce a fine particle spray containing the active agent. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

The aerosol, whether formed from solid or liquid particles, may be produced by the aerosol generator at a rate of from about 10 to 150 litres per minute, more preferably from about 30 to 150 litres per minute, and most preferably about 60 litres per minute. Aerosols containing greater amounts of medicament may be administered more rapidly.

Dosage levels of the order of from about 0.05 mg to about 140 mg per kilogram of body weight per day are useful in the treatment of the above-indicated conditions (about 2.5 mg to about 7 g per patient per day). For example, inflammation may be effectively treated by the administration of from about 0.01 to 50 mg of the compound per kilogram of body weight per day (about 0.5 mg to about 3.5 g per patient per day).

The invention will now be further described by way of reference only to the following non-limiting examples. It should be understood, however, that the examples following are illustrative only, and should not be taken in any way as a restriction on the generality of the invention described above. In particular, while the invention is described in detail in relation to a specific asthma formulation, it will be clearly understood that the findings herein are not limited to this formulation. For example, other formulations for other airway disease may be produced using the techniques herein described as long as they comprise the harmonic disclosed.

### Example 1

### Nutrient Formulation Preparation

The applicant produced a nutrient formulation for the treatment of asthma as follows:

Ascorbic acid from about 250 to 350 mg

Calcium from about 200 to 290 mg

Magnesium from about 20 to 25 mg

Zinc from about 20 to 25 mg

Selenomethionine from about 0.02 to 0.1 mg

Na Bicarbonate from about 330 to 400 mg

Boron from a homeopathic source between 1× and 20×

Probiotic Bacteria between 1 to 10¹¹ cfu per gm.

These ingredient were blended together. Daily dosages could range from between 0.125 mg for infants up to about 6 grams for adults. In order to produce a liquid formulation the appropriate dosage amounts of the formulation was mixed with between 400 to 1000 ml of water and 2% surfactant was added.

The formulation was then vortexed for 45-90 minutes at 30-120 rpm as described above to produce the fundamental quantum harmonic of between 20 to 50 Hz as measured by Protek multifunction counter 9100 frequency meter.

Table 1 shows a series of frequency measurements taken by protek multifunction counter 9100 frequency meter of liquids prior to agitation and after agitation.

TABLE 1
EXAMPLES OF FREQUENCIES (“Freq”) OF DIFFERENT FLUID MEDIUMS

| | | | End | | | | End | |
|---|---|---|---|---|---|---|---|---|
| | Initial | Vortex | Vortex | Vortex | Time | Succussion | Succussion | Time |
| Material | Freq | Freq | Freq | Speed | Vortex | Freq | Freq | Succussion |
| Water | 0 | 9.75 | 249 | 18 | 60 | 9.8 | 31.8 | 60 |
| Milk | 6.6 | 9.81 | 227 | 18 | 60 | 9.6 | 31.01 | 60 |
| Nutrient | 5.9 | 9.819 | 239 | 18 | 60 | 9.85 | 31.65 | 55 |

The experimental data shown in Table 2 indicates that energy was imparted into the liquid medium during the vortexing and agitating process. This is further proven by the measurement of frequencies of the liquid medium before and after processing which show improvements of >100%. All frequencies were measured by protek multifunction counter 9100 frequency meter method.

Bioresonance testing was completed on the fluid mediums of H2O, milk and liquid nutrient formulation. These were tested by the Bioresonance Method of Schimmel (Schimmel, H, 1986, Bioenergetic Regulatory Techniques VEGA Gieshaber GmbH & Co, Am Hohenstein 113 PO 1142D 7-622 Scitach Germany). Increases in resonance show improvements of between 20 and 40%. The optical density was measured by Englehart colorimeter and showed improvements of >%75.

The frequencies of the post agitation frequencies remained constant at a range of between 20 and 50 Hz and revealed that the fundamental harmonic of the agitated materials H2O, milk and nutrient formulation to be maintained and therefore a stable biomorphogenic end product attained.

Once produced the formulation was then ready to be administered to patients as a medicine in order to stimulate certain enzymes of the body which when sufficiently active are capable of clearing from the body=numerous accumulated undesirable non-end product metabolites and toxins.

TABLE 2
EXAMPLES OF BIORESONANCE AND
OPTICAL CHARACTERISTICS

| | | Increase | | |
|---|---|---|---|---|
| | Bioresonance | Bioresonance | Pre-Optical | Post-Optical |
| | % | % | Characteristic | Characteristic |
| 45 | 85 | 40 | 1.1 | 0.4 |
| 80 | 100 | 20 | na | na |
| 80 | 100 | 20 | 1.9 | 1.1 |

### Example 2

### Asthma Clinical Trial

109 candidates with asthma were selected at random and trailed on the nutrient composition described in Example 1 for a period of 1 month. Over a 4 week period Symptom charts noting frequency of cough, wheeze and shortness of breath were kept by the candidates. Weekly questionnaires denoting drug dosage and frequency of symptoms were also returned to the sponsor. Comparisons of symptoms and drug dosage were made comparing pre and post supplementation with the nutrient composition.

Some of the symptom severities were recorded using fractional values (e.g. 0.25) instead of the categories of Nil (0), Mild (1), Moderate (2) and Severe (3). To make use of these entries, the severity values were rounded to the nearest integer using the following scheme:

If 0 ≦ severity < 0.5 then severity = 0.
If 0.5 ≦ severity < 1.5 then severity = 1.
If 1.5 ≦ severity < 2.5 then severity = 2.
If 2.5 ≦ severity < 3.0 then severity = 3.

The frequency and percentage distributions of the reported bronchodilator use at enrolment and after four weeks of treatment were examined to get an indication of whether a change had occurred.

Cross tabulations of the symptom severities at enrolment and after the four weeks of treatment were performed to describe how the severities had changed and to what degree over this period.

Differences in bronchodilator use before and after the treatment period we compared using paired t tests. The symptom severity values are ordinal variables so the Wilcoxon rank sum test was used to determine whether the baseline and week four symptom severity distributions differed primarily in location. That is whether one of the distributions has been shifted left or right of the other.

One-sided tests of significance were used since it was expected that the treatment would improve the severity of the symptoms and reduce the amount of bronchodilators used by the subjects. All tests of statistical significance were made at the 5% level.

### Symptom Severity Cross Tabulations

### Coughing

From Table 3 67.9% (74 of 109) subjects had some reduction in the severity of their coughing after four weeks of the treatment, 27.5% (30 of 109) remained the same and 4.6% (5 of 109) got worse. This was likely due to an inadequate daily dose and also the winter influenza outbreak.

Among those who initially had severe coughing after the treatment, 37.1% (13 of 35) did not report any coughing, 37.1% (13 of 35) reported mild coughing, 14.3% (5 of 35) reported coughing of moderate severity and 11.4% (4 of 35) reported no improvement (Table 3).

TABLE 3
CROSS TABULATION OF COUGH SEVERITY
AT ENROLLMENT BY COUGH SEVERITY AFTER
FOUR WEEKS OF TREATMENT

| Cough | Cough Severity After Treatment | | | | | | | | |
|---|---|---|---|---|---|---|---|---|---|
| severity at | Nil | | Mild | | Moderate | | Severe | | Total |
| enrolment | N | % | N | % | N | % | N | % | N |
| Nil | 13 | 100 | 0 | 0 | 0 | 0. | 0 | 0.00 | 13 |
| Mild | 13 | 50 | 9 | 34.6 | 2 | 7.7 | 2 | 7.69 | 26 |
| Moderate | 16 | 45.7 | 14 | 40.0 | 4 | 11.4 | 1 | 2.86 | 35 |
| Severe | 13 | 37.1 | 13 | 37.1 | 5 | 14.3 | 4 | 11.43 | 35 |
| Total | 55 | | 36 | | 11 | | 7 | | 109 |

### Shortness of Breath

A similar pattern was found for shortness of breath and wheezing.

For shortness of breath, 78.9% (86 of 109) reported a reduction in severity, 18.3% (20 of 109) reported no change and 2.8% (3 of 109) reported getting worse (Table 4).

For those who initially reported having a severe shortness of breath, 28.8% (11 of 41) reported no shortness of breath after four weeks of treatment, 39.0% (16 of 41) had moved to the mild category, 19.5% (8 of 41) were in the moderate category and 14.6% (6 of 41) reported no change (Table 4).

TABLE 4
CROSS TABULATION OF SHORTNESS OF BREATH SEVERITY
AT ENROLMENT BY SHORTNESS OF BREATH SEVERITY
AFTER FOUR WEEKS OF TREATMENT

| Shortness | | | | | | | | | |
|---|---|---|---|---|---|---|---|---|---|
| of breath | Shortness of breath severity after treatment | | | | | | | | |
| severity at | Nil | | Mild | | Moderate | | Severe | | Total |
| enrolment | N | % | N | % | N | % | N | % | N |
| Nil | 3 | 100.00 | 0 | 0.00 | 0 | 0.00 | 0 | 0.00 | 3 |
| Mild | 11 | 57.89 | 5 | 26.32 | 2 | 10.53 | 1 | 5.26 | 19 |
| Moderate | 21 | 45.65 | 18 | 39.13 | 6 | 13.04 | 1 | 2.17 | 46 |
| Severe | 11 | 26.83 | 16 | 39.02 | 8 | 19.51 | 6 | 14.63 | 41 |
| Total | 46 | | 39 | | 16 | | 8 | | 109 |

### Wheezing

For the wheezing symptom, 68.8% (75 of 109) showed some improvement in symptoms, 28.4% (31 of 109) did not change and 2.8% (3 of 109) were worse off (Table 5).

For those initially in the severe wheezing category, 37.5% (12 of 32) reported no wheezing after treatment, 34.4% (11 of 32) were in the mild group, 12.5% (4 of 32) had moved to the moderate group and 15.6% (5 of 32) reported no improvement. (Table 5).

TABLE 5
CROSS TABULATION OF WHEEZE SEVERITY
AT ENROLMENT BY WHEEZE SEVERITY AFTER
FOUR WEEKS OF TREATMENT

| Wheeze | Wheeze severity after treatment | | | | | | | | |
|---|---|---|---|---|---|---|---|---|---|
| severity at | Nil | | Mild | | Moderate | | Severe | | Total |
| enrolment | N | % | N | % | N | % | N | % | N |
| Nil | 12 | 100 | 0 | 0 | 0 | 0 | 0 | 0 | 12 |
| Mild | 13 | 50 | 12 | 46.2 | 1 | 3.9 | 0 | 0 | 26 |
| Moderate | 18 | 46.2 | 17 | 43.6 | 2 | 5.1 | 2 | 5.1 | 39 |
| Severe | 12 | 37.5 | 11 | 34.4 | 4 | 12.5 | 5 | 15.6 | 32 |
| Total | 55 | | 40 | | 7 | | 7 | | 109 |

### Bronchodilator T Test

From the paired t tests on the amount of bronchodilators doses used, a significant decrease in the amount of Ventolin taken via puffer (p-value=0.0007) and nebuliser (p-value=0.0176), as well as Seretide (p-value=0.0084) and Flixotide (p-value=0.0400) after the four week treatment period (Table 6).

An examination of the usage data for the other bronchodilators in the data set showed that only a small proportion of the subjects (at most 15%) used these other products/substances. With such small numbers meaningful analyses could not be performed on these other data.

TABLE 6
PAIRED T TEST RESULTS FOR STATISTICALLY
SIGNIFICANT CHANGES IN BRONCHODILATOR USE
BETWEEN ENROLMENT AND AFTER TREATMENT

| Bronchodilator | DF | t Value | Pr > \|t\| |
|---|---|---|---|
| Ventolin | 107 | −3.49 | 0.0007 |
| Ventolin Nebuliser | 108 | −2.41 | 0.0176 |
| Seretide | 108 | −2.69 | 0.0084 |
| Flixotide | 108 | −2.08 | 0.0400 |

°* Please note, these values are statistically significant at the 5% level.

Ventolin puffer use fell from a mean of 3.8 doses at enrolment to 1.7 after four weeks of treatment. The use of Seretide, Flixotide and Ventolin via nebuliser also fell after four weeks of treatment by smaller amounts in absolute terms, however, the proportional change was similar (Table 7).

TABLE 7
MEAN AND MEDIAN NUMBER OF DOES
OF BRONCHODILATOR USE BETWEEN ENROLMENT
AND AFTER TREATMENT

| | Mean | Mean |
|---|---|---|
| Bronchodilator | (enrollment) | (week 4) |
| Ventolin | 3.8 | 1.7 |
| Ventolin Nebuliser | 0.7 | 0.2 |
| Seretide | 1.0 | 0.6 |
| Flixotide | 0.5 | 0.3 |

### Symptom Severity Non-Parametric Tests

### Cough

The Wilcoxon tests suggest that one of the distributions has a significantly higher cough severity scores than the other (Norm approx Z=7.5365, p-value <0.0001) (Table 8). Using the information from Table 3 it can be seen that the severities at the time of enrolment were more severe than the values after the four weeks of treatment.

TABLE 8
WILCOXON TWO SAMPLE TEST RESULTS
FOR CHANGES IN COUGH SEVERITY

| Statistic | 15320.5 |
|---|---|
| Normal Approximation | |
| Z | 7.5365 |
| One-Sided Pr > Z | <.0001 |
| Two-Sided Pr > \|Z\| | <.0001 |
| Student's t Approximation | |
| One-Sided Pr > Z | <.0001 |
| Two-Sided Pr > \|Z\| | <.0001 |

Z includes a continuity correction of 0.5

### Shortness of Breath

Similarly the Wilcoxon test for shortness of breath indicated that there was a statistically significant difference in the distributions of severities at enrolment and after four weeks for this symptom (Norm approx Z=8.7827, p-value <0.0001) (Table 9). From Table 4 it can be seen that the severities reported at enrolment were more severe than after the treatment period.

TABLE 9
WILCOXON TWO SAMPLE TEST RESULTS FOR CHANGES
IN SHORTNESS OF BREATH SEVERITY

| Statistic | 15891.5 |
|---|---|
| Normal Approximation | |
| Z | 8.7827 |
| One-Sided Pr > Z | <.0001 |
| Two-Sided Pr > \|Z\| | <.0001 |
| Student's t Approximation | |
| One-Sided Pr > Z | <.0001 |
| Two-Sided Pr > \|Z\| | <.0001 |

Z includes a continuity correction of 0.5

### Wheeze

There were statistically significant differences in the distribution of severities for wheezing between the initial severities and those recorded after four weeks. With the information from Table 5 it can be seen in Table 10 that there was a statistically significant improvement in the severities of wheezing after four weeks of treatment.

TABLE 10
WILCOXON TWO SAMPLE TEST RESULTS
FOR CHANGES IN COUGH SEVERITY

| Statistic | 15492.5 |
|---|---|
| Normal Approximation | |
| Z | 7.928 |
| One-Sided Pr > Z | <.0001 |
| Two-Sided Pr > \|Z\| | <.0001 |
| Student's t Approximation | |
| One-Sided Pr > Z | <.0001 |
| Two-Sided Pr > \|Z\| | <.0001 |

Z includes a continuity correction of 0.5

### Summary/

From these data it appeared that the treatment was associated with a statistically significant decrease in the use of Ventolin (puffer and nebuliser), Seretide and Flixotide, and that is also associated with a significant decrease in the severity of coughing, wheezing and shortness of breath after four weeks of treatment.

### Example 3

### Homeopathic—Biomorphogenic Medicine

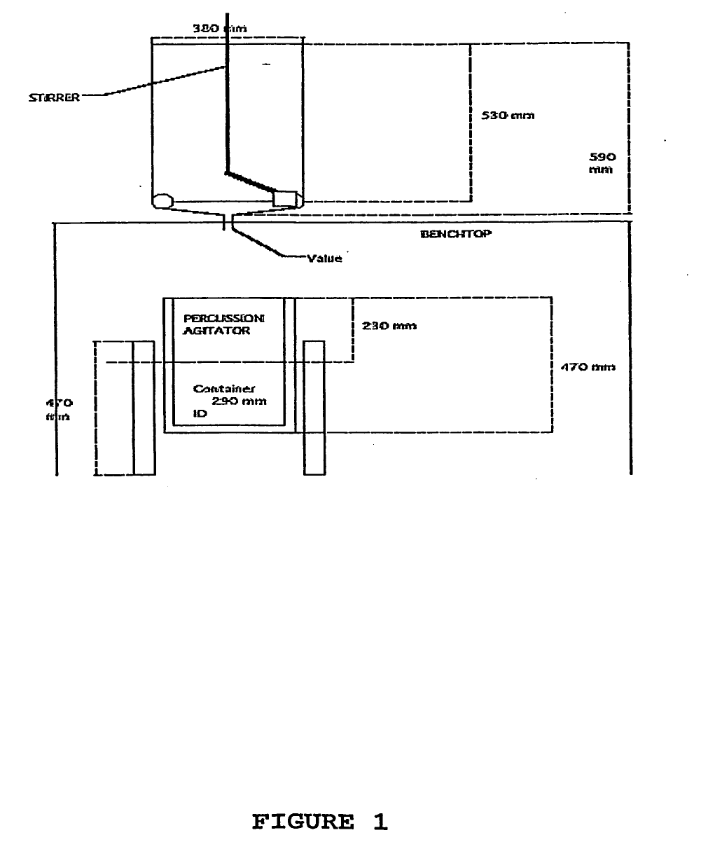

A 1 ml aliquot of the nutrient formulation described in Example 1 was diluted with 9 ml of diluent to produce 10 ml of 1× attenuation. This was then vortexed and rotated as described elsewhere above for 45-90 minutes. See FIG. 1.

A further dilution of the nutrient formulation was made by taking 1 ml of the 2× attenuation and succussed with 9 mls of diluent to produce 10 ml of 3× attenuation and so on. This may be repeated until the desired potency is acquired.

Should a liquid formulation be required, suspension in alcohol is the specified menstruum for the final decimal or centesimal attenuation when intended for medical purposes. The amount of alcohol will vary from between 24-60% depending on the desired potency.

There is a unique synergy between all constituents in the present nutrient formulation. This promotes rapid absorption of nutrient in the gut lining. This has been shown by the applicant to occur within 10-30 seconds of taking the powder orally.

### Example 4

### Incorporation of Nutrient Formulation in Food

The nutrient formulation may be utilised as a medical food to regulate free radical scavenging and liver detoxification by maintaining a balanced formula of key nutrients required for correct functioning of cytochrome P450 enzyme pathways of the consumer of the formulation. The nutrient formulation disclosed in Example 1 may be added to liquids such as milk, powdered milk, water or juice to supplement the drink.

1-15. (canceled)

16. A method of producing a nutrient formulation or composition for treating asthma in a human subject in need of such treatment, said method comprising:
(a) providing a mixture of probiotic bacteria and at least one other ingredient selected from the group consisting of ascorbic acid, calcium, magnesium, zinc, selenomethionine, sodium bicarbonate and boron;
(b) vortexing said mixture for between about 45 and about 90 minutes to impart between about 50 to about 100 joules per second to said mixture to produce a vortexed mixture;
(c) agitating said vortexed mixture at a rate of between about 50,000 to about 65,000 kJ/mole at an angle of between about 10 degrees to about 90 degrees to impart a harmonic frequency in said vortexed mixture of between 20 to 50 Hz.
thereby producing said nutrient formulation for treating asthma.

17. The method of claim 16, wherein the mixture further comprises a drug, a peptide, a protein, a carbohydrate, a nucleoprotein, a mucoprotein, a lipoprotein, a synthetic polypeptide, a steroid, a nucleic acid, a nucleotide, a nucleoside, an oligonucleotide, a gene, a lipid, a hormone, a vitamin, a mineral, an antioxidant, a chemotherapeutic agent, a hormone, an antibiotic, an antiviral agent, an antifungal agent, an antiproliferative agent, an antihistamine, an anticoagulant, a non-steroidal antiinflammatory compound or a combination thereof.

18. The method of claim 16, wherein the mixture comprises, per gram:
(a) from 1 and up to 10¹¹ colony forming units of said probiotic bacteria;
(b) about 250 to about 350 mg ascorbic acid;
(c) about 200 to about 290 mg calcium;
(d) about 20 to about 25 mg magnesium;
(e) about 20 to about 25 mg zinc;
(f) about 0.02 to about 0.1 mg selenomethionine; and
(g) about 330 to about 400 mg sodium bicarbonate.

19. The method of claim 17, wherein the mixture comprises, per gram:
(a) from 1 and up to 10¹¹ colony forming units of said probiotic bacteria;
(b) about 250 to about 350 mg ascorbic acid;
(c) about 200 to about 290 mg calcium;
(d) about 20 to about 25 mg magnesium;
(e) about 20 to about 25 mg zinc;
(f) about 0.02 to about 0.1 mg selenomethionine; and
(g) about 330 to about 400 mg sodium bicarbonate.

20. A method of treating asthma in a human subject, comprising administering orally to a subject in need thereof a formulation or composition produced by the method of claim 16.

21. A method of treating asthma in a human subject, comprising administering orally to a subject in need thereof a formulation or composition produced by the method of claim 17.

22. A method of treating asthma in a human subject, comprising administering orally to a subject in need thereof a formulation or composition produced by the method of claim 18.

23. A method of treating asthma in a human subject, comprising administering orally to a subject in need thereof a formulation or composition produced by the method of claim 19.
